(19) United States
(12) Patent Application Publication
(10) Pub. No.: US 2024/0262871 A1
(43) Pub. Date: Aug. 8, 2024

(54) FUSION PROTEIN FOR DETECTING NEUROSYPHILIS AND KIT THEREOF

(71) Applicant: Dermatology Hospital, Southern Medical University, Guangzhou (CN)

(72) Inventors: Wujian KE, Guangzhou (CN); Xiaohui Zhang, Guangzhou (CN); Liuyuan Wang, Guangzhou (CN); Yujiao Wu, Guangzhou (CN); Xinying Leng, Guangzhou (CN); Sheng Ke, Guangzhou (CN)

(73) Assignee: Dermatology Hospital, Southern Medical University, Guangzhou (CN)

(21) Appl. No.: 18/185,979

(22) Filed: Mar. 17, 2023

(30) Foreign Application Priority Data
Feb. 8, 2023 (CN) 202310081049.3

(51) Int. Cl. C07K 14/20; C12N 15/63; C12Q 1/689

(52) U.S. Cl. CPC C07K 14/20; C12N 15/63; C12Q 1/689; C07K 2319/61

(57) ABSTRACT

A fusion protein for detecting neurosyphilis and a kit thereof are provided in this disclosure. It is unexpectedly found in this disclosure that detecting the Nichols Houston strain TP0136 antibody level for diagnosis of neurosyphilis makes up a blank of diagnosis and detection methods of the neurosyphilis. Using molecular biology gene cloning and expression technologies, a syphilis Nichols Houston strain TP0136 recombinant protein is obtained, a luciferase immunoprecipitation method for detecting a Nichols Houston strain TP0136 antibody is established, and a kit is assembled. Such a kit has such unexpected technical effects of high sensitivity and high prediction accuracy, and has advantages of large detection throughput, simple operation and easy popularization.

### CROSS-REFERENCE TO RELATED APPLICATION

This Non-provisional application claims priority under 35 U.S.C. § 119(a) to Chinese Patent Application No. 202310081049.3, filed on 8 Feb. 2023, the entire contents of which is hereby incorporated by reference in its entirety.

### INCORPORATION BY REFERENCE OF SEQUENCE LISTING

A Sequence Listing is provided herewith as an xml file, “2347485.xml” created on Jun. 28, 2023 and having a size of 7,030 bytes. The content of the xml file is incorporated by reference herein in its entirety.

### TECHNICAL FIELD

The disclosure relates to the field of biomedicine, in particular to a detection kit.

### BACKGROUND ART

Existing diagnosis of neurosyphilis mainly relies on laboratory detection of cerebrospinal fluid. As a “gold standard” for cerebrospinal fluid diagnosis of neurosyphilis, specificity of a venereal disease research laboratory (VDRL) test is 90% with a sensitivity of only 50%, so the negative result of VDRL test cannot exclude neurosyphilis. In addition, application of this method in diagnosis of the neurosyphilis is further limited due to a biological false positive problem (that is, the positive in the VDRL test occurs for nonsyphilis patients). However, other commonly used methods for cerebrospinal fluid detection are not sensitive or specific, which makes it difficult to diagnose the neurosyphilis and with a high misdiagnosis rate. A retrospective study showed that a misdiagnosis rate of detecting the neurosyphilis in terms of cerebrospinal fluid was as high as 84.6% (126/149). Therefore, a new method for diagnosis of neurosyphilis is urgently needed in clinic, so as to avoid excessive lumbar puncture and provide a rapid and simple detection method for diagnosis and treatment of neurosyphilis.

TP0136 protein heterogeneity can be used to identify the potential of TP subspecies and to distinguish TPA, TPE, FB and TEN. TP0136 molecular typing can facilitate further improvement of traditional TPA typing methods and help to explore genotype and clinical diagnosis, development and prognosis and drug resistance monitoring.

A luciferase immunoprecipitation system (LIPS) is a detection technique for determining unknown antibodies in serum by fusion expression of luciferase and antigen in mammalian cells. The antigen and luciferase are subjected to the fusion expression in the LIPS, and an antibody in serum is specifically combined with the antigen, and the antibody is then combined with a large particle matter, protein A/G plus agarose, which are formed into a complex. An antibody level in the serum can be evaluated by analyzing fluorescence intensity of the luciferase on the complex when it interacted with a substrate. Expression in the mammalian cells of the LIPS system may form a spatial conformation antigen, which can improve detection efficiency. Meanwhile, the antigen used in the system does not need to be purified, which simplifies experimental steps. Therefore, the LIPS system is widely used for detection of many autoimmune diseases and pathogens. However, the LIPS is operated with a single tube, which cannot achieve high-throughput detection, and experiments are time-consuming.

The disclosure needs to solve above technical problems and provides a rapid and simple detection kit for the diagnosis and treatment of neurosyphilis.

### SUMMARY

In order to solve shortcomings of related art, an object of the disclosure is to provide a fusion protein for detecting neurosyphilis and a kit thereof. According to the disclosure, using molecular biology gene cloning and expression technologies, a syphilis Nichols Houston strain TP0136 recombinant protein is obtained, a luciferase immunoprecipitation method for detecting a Nichols Houston strain TP0136 antibody is established, and a kit is assembled, in which neurosyphilis is distinguished based on a Nichols Houston strain TP0136 antibody level of neurosyphilis infected persons, which facilitates guiding of clinical rational drug use, avoiding excessive lumbar puncture, and provides a diagnostic kit with high prediction accuracy and sensitivity.

A fusion protein for detecting neurosyphilis is a fusion protein for expressing a gene fragment of a Nichols Houston strain TP0136 antigen and a bioluminescence reporter gene. The gene fragment of the Nichols Houston strain TP0136 antigen is a fragment of a Gene group sequence with genbank ID of 3322413, located from Start: 156807 to Stop: 158276 and with Gene Length: 1470. The gene fragment of the Nichols Houston strain TP0136 antigen is obtained by taking DNA of Treponema pallidum Nichols Houston strain as a sequence template and taking the Nichols Houston strain TP0136 as a primer.

For the fusion protein for detecting the neurosyphilis, the sequence template SEQ01 is 16 to 1470 sites of the gene fragment of the Nichols Houston strain TP0136 antigen; and an upstream primer SEQ ID NO: 2 is 5′-ATGACGTGCGATTTCACTGG-3′, and a downstream primer SEQ ID NO: 3 is 5′-CTCGCGGTTCCAGGAGCACG-3′. For the fusion protein for detecting the neurosyphilis, the bioluminescence reporter gene is a luciferase reporter gene.

For the fusion protein for detecting the neurosyphilis, the luciferase reporter gene is a dual-luciferase reporter gene.

For the fusion protein for detecting the neurosyphilis, the fusion protein is obtained by expression of a recombinant plasmid obtained by respective enzyme digestion and ligation of the gene fragment of the Nichols Houston strain TP0136 antigen and a pNLF1 vector plasmid of a NanoLuc luciferase gene.

For the fusion protein for detecting the neurosyphilis, the enzyme digestion is EcoR I and Xba I double digestion, with a ligase being a T4 ligase.

For the fusion protein for detecting the neurosyphilis, the expression is made by following steps:
1) transforming the recombinant plasmid into an E. coli DH5α competent cell, and then incubating, inoculating, culturing, PCR identifying and sequencing;
2) performing cell transfection on the recombinant plasmid by using a liposome-mediated method; and
3) performing cell lysis on transfected cells and then collecting supernatant which is diluted to obtain the fusion protein.

A fusion protein kit for detecting neurosyphilis includes: (1) a sample diluent; (2) a fusion protein solution consisting of NanoLuc luciferase and a Treponema pallidum Nichols Houston strain TP0136 antigen; (3) a proteinA/G coated enzyme-labeled plate; (4) a washing liquid; and (5) a luciferase substrate.

In the fusion protein kit for detecting the neurosyphilis, the Treponema pallidum Nichols Houston strain TP0136 antigen is extracted by inoculating Treponema pallidum Nichols Houston strain into testes of 3-month-old New Zealand rabbits, and then extracting a genome of a testicular tissue sample with a DNA extraction kit.

In the fusion protein kit for detecting the neurosyphilis, the sample diluent is 2% skim milk.

In the fusion protein kit for detecting the neurosyphilis, the washing liquid is with a formula of: 8 g of NaCl, 0.2 g of KCl, 3.63 g of Na₂HPO₄·12H₂O, and 0.24 g of KH₂PO₄, with a volume being adjusted to 1 L by adding water. This is just an example, as long as it can be applied to the cleaning solution of the present disclosure, it is within a protection scope of the present disclosure.

In the fusion protein kit for detecting the neurosyphilis, the luciferase substrate is a furimazine luciferase substrate from Promega Corp. This is just an example, and the luciferase substrate can also be other products, so long as it can emit bioluminescence during reaction with the luciferase, it is suitable for the present disclosure.

### Terminology

Fusion protein: an expression product by combination of two genes and obtained by DNA recombination technologies.

PCR amplification: Polymerase chain reaction (PCR) is a molecular biology technique used to amplify specific DNA fragments, which can be regarded as a special DNA replication in vitro, with a biggest feature of the PCR that it can greatly increase trace amount of DNA.

The disclosure has advantages as follows.

It is unexpectedly found in this disclosure that detecting the Nichols Houston strain TP0136 antibody level for diagnosis of neurosyphilis makes up a blank of diagnosis and detection methods of the neurosyphilis. The method has unexpected technical effects of high sensitivity and high prediction accuracy, has advantages of large detection throughput, simple operation and easy popularization, and has market value.

A luciferase immunoprecipitation method is adopted in the disclosure, which can be used for the diagnosis of neurosyphilis according to fluorescence intensity. The method is rapid, simple, and is with a high sensitivity and high signal-to-noise ratio, and a stable and reliable measured value.

### DETAILED DESCRIPTION

The disclosure will be described in the following in detail with reference to specific embodiments.

Our previous research found that TP0136 presents genetic heterogeneity in different strains of TP and TPA. Taking advantage of this feature, in this document, a new TP molecular typing method is established by studying TP0136 protein heterogeneity among TPA, TPE, FB and TEN strains.

### I. Materials and Methods

1. Sample collection and DNA extraction: 23 cases of blood, cerebrospinal fluid or chancre exudation from STD clinics in Guangdong Province from January 2015 to December 2018 were collected. TRUST and TPPA serum tests were positive for all patients. 23 clinical samples were numbered from GD001 to GD023. (1)Blood sample: 2 mL of venous blood of a subject was collected and placed in an EDTA anticoagulant tube; (2) Cerebrospinal fluid sample: 1 mL of cerebrospinal fluid was collected in a sterile tube by lumbar puncture; (3) Chancre exudation sample: a surface of chancre is washed with normal saline, a surface of a lesion is gently scraped with a blunt knife and then is squeezed in such a way to avoid blood contamination of the exudate. 0.5 ml of blood, cerebrospinal fluid or chancre exudate are mixed with 0.5 ml of 2× cell lysate buffer (20 mM of Tris-HCl, 0.2 M of EDTA, 1% of SDS), and a QIAamp DNA Mini kit (QIAGEN) is applied to extract DNA for subsequent steps. Steps of extracting DNA are strictly in accordance with instructions, and the extracted DNA is cryopreserved at −20° C.

2. Main reagents and instruments: a TPPA kit (purchased from fuji company), a TRUST kit (purchased from Shanghai RongSheng company), a QIAamp DNA Mini kit (purchased from Qiagen company, Germany) and T100PCR instrument (purchased from Bio-Rad company, USA).

3. Sequence analysis and primer design of TP0136: amino acid and nucleic acid sequences of TP0136 were searched from genbank, and 9 Treponema TPA (Nichols Houston, Nichols Seattle, Nichols Dallas, DAI-1, Bal73-1, Seattle81-4, Chicago, MexicoA and SS14) and 3 TPE (Samoa D, CDC2 and Gauthier) that did not cause human sexually transmitted diseases, one unclassified anthropoid Treponema (Fribourg-Blanc) and one TEN (Bosnia A) were subjected to multiple sequence alignment, and analyzed using a BioEdit 7.1 software. A primer is designed for a TP0136 open reading frame (ORF) using a Primer Premier 5.0 software. A TP0136 signal peptide was predicted using a SignalP 4.1 Server software, and the primer for the TP0136 open reading frame did not contain a signal peptide. Due to difference in a COOH end of a TP0136 protein, two pairs of different primers were designed to correspond to different COOH ends of TP0136 proteins of different strains.

A first pair of upstream primers for Tp0136 is SEQ ID NO: 2: 5′-ATGACGTGCGATTTCACTGG-3′; and a first pair of downstream primers for Tp0136 is SEQ IDNO: 3: 5′-CTC GCGGTTCCAGGAGCACG-3′, with an amplified product of a size of 1389 bp, and target strains of Nichols Houston, Bal73-1, Seattle81-4, Chicago and MexicoA SS14.

A second pair of upstream primers for Tp0136 is SEQ ID NO: 4: 5′-ATGACGTGCGATTTCACTGG-3′; and a second pair of downstream primers for Tp0136 is SEQ ID NO: 5: 5′-ACTACGTAGATTTTCTGCAC-3′, with an amplified product of a size of 1260 bp, and target strains of Nichols Seattle, DAI-1 and Nichols Dallas.

4. PCR amplification of TP0136 gene: DNA of 23 clinical isolates of TP0136 was amplified by conventional PCR. 50 μL of PCR amplification reagent contains: 5 μL of DNA sample to be detected, 200 μM of dNTPs, 5 μL of 10×Go Taq PCR buffer (Promega), 1.5 mM of MgCl₂, 0.6 μM of TP0136 primer and 0.5 U of hot-start Taq PCR polymerase (Promega). Amplification conditions are as follows: predegeneration at 95° C. for 10 min; degeneration at 95° C. for 1 min, annealing at 60° C. for 2 min and extending at 72° C. for 1 min, with a total of 45 cycles; and extending at 72° C. for 10 min. The amplified PCR products were identified by 1.5% agarose gel electrophoresis. After products amplified by PCR were purified and recovered by an ExoSAP-IT PCR product purification kit (Affymetrix, Santa Clara, CA), ORFs of 23 TPA strains were sequenced by two-way DNA sequencing (Shanghai Sangon Co., Ltd.).

5. Three-gene joint typing method: a number of repeated sequences of TPA acidic repeat protein (Arp) gene, a type of Restriction Fragment Length Polymorphism (RFLP) after analysis of repeat gene (T. pallidum repeat gene, Tpr) and Mse I enzyme digestion of gene and a type of a TP0548 gene sequence were combined to establish a three-gene typing method with Arp/Tpr/TP0548 as a core. A number of repeated sequences of Arp gene with 60 base pairs was analyzed by a gel electrophoresis analysis software, and compared with a Nichols standard strain with 14 repeated sequences, which can be divided into “2 to 22” types. Tpr II gene is subjected to RFLP analysis after Mse I enzyme digestion and is compared with the “a to p” types reported in previous literature. After a purified TP0548 gene PCR product was sequenced by Shanghai Sangon Co., Ltd., sequencing results were compared with the “a to i” types using the BioEdit7.1 software.

### II, Results

### Sequence Analysis Results of 14 Treponema TP0136

After multiple sequence alignment, the results showed that there were a large number of gene insertions, deletions or partially deletions in 14 TP0136 amino acid sequences of TP, which indicated high heterogeneity of TP0136. TP0136 amino acid sequences of three TPEs (Samoa D, CDC2 and Gauthier) which did not cause human sexually transmitted diseases were different, and could be divided into three types, represented by types I to III. TEN (Bosnia A) can be significantly distinguished from TPE and TPA, and is represented by a type IV. Nine TPAs causing the human sexually transmitted diseases can be divided into six types, represented by types V to X. Except CDC2 and FB in TPE that cannot be completely distinguished, other TPE and FB strains can be distinguished, see Table 1. From a 100th site of amino acid, there are obvious differences among types I to III. At a 108th site, Samoa D is with isoleucine, while CDC2, FB and Gauthier are with serine. At a 166-th site, Gauthier lacks serine compared with CDC2 and FB. Compared with a type VI, a type V in TPA lacks a set of repeat sequences. TP0136 amino acid sequences of Dal-1 and Nichols Seattle are identical, which is divided into the type VI. TP0136 amino acid sequences of Nichols Houston, Chicago and Bal73-1 are identical, which is divided into a type VII. TP0136 amino acid sequences of the other three strains (Seattle 81-4, SS14 and MexicoA) are different, and are expressed by types VIII to X (Table 1).

TABLE 1
Typing of 14 treponema pallidum strains

| Type | Strain Name |
|---|---|
| I | Samoa D |
| II | CDC2, FB |
| III | Gauthier |
| IV | Bosnia A |
| V | Nichols Dallas |
| VI | Dal-1 and Nichols Seattle |
| VII | Nichols Houston, Bal73-1, and Chicago |
| VIII | Seattle81-4 |
| IX | SS14 |
| X | MexicoA |

### Comparative Analysis of Tp0136 Sequences

Sequence identity and similarity of Nichols strain and SS14 strain of T.pallidum were 92% and 94%, respectively. Although these calculations are based on recently discovered genome sequences with a large number of errors, they still hold true when comparing sequences from corrected genomes. Our investigation on Tp0136 genetic diversity was extended to seven other T. pallidum strains (Nichols Houston, Nichols Seattle, Nichols Dallas, Dal-1, Mexico, Bal73-1, Seattle81-4 and SS14). For all of these strains, including those with genome availability [33, 34, 35, 36], Tp0136 was re-sequenced in the laboratory. Results showed that in addition to two reported mutations, four new variants of Tp0136 were found, which confirmed inter-plant variability of Tp0136. Predictive results of all identified amino acid sequence with TP0136 variants (excluding aa 1 to 31, belonging to the protein signal peptide) showed that TP0136 sequences of Nichols Seattle and Dal-1 were identical. A Nichols Dallas sequence is the same as a Nichols Seattle sequence, but there are 32 amino acid repeat sequences missing in a Nichols Dallas protein. Diversity between Nichols Seattle/Dallas and Nichols Houston sequences is due to insertion of 198 bp into the Nichols Seattle/Dallas gene sequence, which also leads to frame shift and premature termination of protein translation. Our research group found earlier that this insertion site contains a donor sequence of T. pallidum tprK gene, and it is known that the donor sequence is recombined into nonreciprocal gene transformation mediated by a gene expression site. Predicted difference in the TP0136 protein sequences between Nichols Seattle and Nichols Houston prompted us to study its influence on fibronectin binding ability of these two variants. Nichols Houston and Bal73-1 were also found to carry a same TP0136 sequence. Except for one amino acid substitution, sequences of MexicoA and SS14 strains are identical. Compared with other isolates, a TP0136 protein of Seattle81-4 is unique.

In the disclosure, the above mechanism is applied to design a neurosyphilis diagnosis kit based on a TP0136 protein of a Nichols Houston strain.

It includes: (1) a sample diluent; (2) a fusion protein solution consisting of NanoLuc luciferase and a Treponema pallidum Nichols Houston strain TP0136 antigen; (3) a proteinA/G coated enzyme-labeled plate; (4) a washing liquid; and (5) a luciferase substrate.

The sample diluent is 2% skim milk.

The fusion protein solution is prepared using a following method:
a, taking the gene sequence of Nichols Houston strain TP0136 antigen as a template, the sequence template being SEQ ID NO: 1: ATGGATACGCAGTATATGAGGCGCCGGGTGTGCACGGTGGTGCGCGCG GTGGTGTGTCTACTCAGCACGAGTTTGCTGACCACGTGCGATTTCACT GGCATCTTTGCGGCAATTCAGTCGGAAGTGCCCATTAAAACGCCGTCC ATCCCGGGGGCGATTTATGGCCTGGTCAAGGCCGGGAGCAAGCTCTAC GCCACCAACGGCCGGCTTTGGGAAAAGGAGCTGAACGGCACTGGGT CGTGGCAGAAAGTGTCTTCCTCGTCCGTTCCCACTGACTCGGATAAAA AGGTTATGAGCATTGCCACCGACGGGAACACGTTCGTCCTCGCCTGCG TGCCTGGCACGGGCGTTTACAAACACTGCGTAAATGGCGCGGGCAGC TCAAGCACCGGCACAACGGCAAGCCCCTCGACTGAAACCTGCTCGCA GCATGCGACGCTCGTGGGGGGAACGTCCAAGCCCTTCTGGCTCGTGC CGGGAGGCACGGGGAATAATGGGAACTGCGGTTGCGGGGGAGGGGG GGGTGGCTCCTCCTCGAGTAGCAGCTCGTGCATTCACATCTGGCTCGT GCCGGGAGGCACGGGGAATAATGGGAACTGCGGTTGCGGGGGAGGG GGGGGTGGCTCCTCCTCGAGTAGCAGCTCGTGCATTCACATTAAGGTA GAAAACACGGACGAACAGTTTCTCGATATGGGTGAGGGGTACGTGGT GACCACCAAGCACCTCTACACCAAAAACGGCTCGTCCAGCGCGGGAC CGGCGCAGTGTCCCGGTGGCGGTGGCGGCGGAGGCAGCAGCGGGGG TGGGGGTTCCTCGGAGTACACCAAAGCTTCCTGTTCCTTTTCCACGCC CATTCTGGCAAGCGTCAGCGACGGGTGCTATCACTACATTCTCACCAA AGAAAAAGTGTACTGCAGAAAGCAGGACACCGCTTCCTCCGCTGCGT CGTCACCAGCCCAGTGTCCCTCTTCCCCTTCTTCTTCTTCCTCCTCCTC GACGAATGCGGGATGCGAGGTGGCGCACGGGGTGGACGACCCGCTGT GTCTTGCGATTTTTAAACACAACGGCTGCGAATACTTGCTCATCGGCG GCAGTCGGGGCTACGGGGAAATAAAGCTGGAAGCGAACTCCAGCGGT ACGAACGGCACCTGCATGCGATTGAAAGAGAGCAATGTGCACAAGAG TCCGGGCCAGTGGGGCGAGTCGAGCCCCACGCCCAAAGCGAGCGCC GAGCAGTATCGGGGCACGGTCGGTCGGTTTGCCGTGCAGAAAATCTA CGTAGTTGAAAAAAATGGCGGTGGGAACGGTGTCGCCGCGGGTGGGG CGGGCTGTCCTGCAAACGCCAGCAGTTCCAGCGGAGGGACCAGCAGC ACGCAGCGTCCAGACCTCTACGCCGCAGTGGGGGAGTCGAGCGACAC CTACACGGGGCTGTGGAAGTTTGACACCACCACGTGCTCCTGGAACC GCGAG, PCR amplification is performed, a PCR amplification product was subjected to gel electrophoresis and nucleic acid purification so as to obtain the gene fragment of the Nichols Houston strain TP0136 antigen.

A primer for PCR amplification is:

an upstream primer:
SEQ ID NO: 2
5′-ATGACGTGCGATTTCACTGG-3′,;
and
a downstream primer:
SEQ ID NO: 3
5′-CTCGCGGTTCCAGGAGCACG-3′,.

b, the obtained gene fragment of the Nichols Houston strain TP0136 antigen and a pNLF1 vector plasmid of a NanoLuc luciferase gene were subjected to EcoR I and Xba I double digestion respectively, and digested products were ligated using a T4 ligase so as to obtain a recombinant plasmid.
c, the recombinant plasmid was transformed into Escherichia coli DH5a, and transgenic positive bacteria was screened by bacterial liquid PCR.
d, the transgenic positive bacteria was reproduced in 200 ml of LB liquid culture medium, and the recombinant plasmid was extracted using a Plasmid extraction kit (Plasmid Plus Midi Kit, Qiagen).
e, quality of the recombinant plasmid extracted in the step d was verified by agarose gel electrophoresis (with a band with no tailing and a correct band size), and purity and concentration of the extracted recombinant plasmid were detected by an ultraviolet spectrophotometer (the purity was high when OD260/OD280 ranged from 1.8 to 2.0, and the plasmid concentration was ng/ml=OD260×50 ng/ml× dilution factor);
f, He La cells were cultured in a 10 cm² cell culture dish, when a cell plating rate reached 80 to 90%, transfecting was performed with a ratio of a liposome nucleic acid transfection reagent (lipofectamine 2000, invitrogen): the recombinant plasmid obtained in the step d of 3 μL: 1 μg, and culturing was made in a 5% CO₂ incubator at 37° C. for 48 to 72 hours, then a cell culture solution was removed; and with 5 ml of a 0.01M phosphate buffer with pH of 7.4 added, the cells were collected into a 15 ml centrifuge tube using a cell scraper, and finally a cell collection solution was ultrasonically treated in an ice bath for 15 to 30 min to obtain a cell lysate, then the cell lysate was centrifuged at 4° C. and 12000 rpm for 15 to 45 min and precipitate was removed to collect supernatant so as to obtain the fusion protein.
g, the obtained fusion protein was diluted with a 0.01M PBS phosphate buffer with a pH of 7.4 to obtain a fusion protein solution. The fusion protein solution was required to have 107 fluorescent units per 10 μL, and is saved at −20° C. for later use.

The fusion protein solution is a diluted fusion protein, and this diluent is also the cell lysate. It is only necessary to ensure a concentration of the fusion protein before the cell lysis, so its dosage is small. After lysis, it is adjusted to a use concentration, and the fusion protein solution with the adjusted concentration is then placed in the kit, so that a detection process for the kit is convenient (there is no need to detect protein concentration).

A preparation method of a proteinA/G coated enzyme-labeled plate is as follows.
a, proteinA/G was dissolved into a concentration of 1 μg/mL with a 0.01M PBS solution with a pH of 7.4.
b, the proteinA/G solution was added into an enzyme-labeled plate at 100 μL/well, the enzyme-labeled plate was covered with a film, and incubating was made at 4° C. for 12 to 16h.
c, the proteinA/G solution was removed, washing was made at 300 μL/well with 0.1% of PBST for three times, and storage was made at 4° C.

A washing liquid was prepared as follows: 8 g of NaCl, 0.2 g of KCl, 3.63 g of Na₂HPO₄·12H₂O, and 0.24 g of KH₂PO₄, with a volume being adjusted to 1 L by adding water and sterilization.

The luciferase substrate is a furimazine luciferase substrate from Promega Corp.

1. Culturing of Treponema pallidum Nichols Houston Strain and Extraction of Genomic DNA.

Treponema pallidum Nichols Houston strains were inoculated into testes of 3-month-old New Zealand rabbits. It is ensured that results of serological test of syphilis in respective rabbits were negative before inoculation, and the rabbits were fed with antibiotic-free feed and water. After 4 weeks, the testes of rabbits were taken and aseptically soaked in sterile physiological saline and washed, and then the testes were cut into pieces with a sterile scissors and placed in an EP tube. With dark-field microscope identification, if white, strong refractive and slender spiral microorganisms were found and with serpentine, rotary, telescopic or other types of slow and regular movement, it can be determined that Treponema pallidum exists.

The tissue samples subjected to identification were handled according to instructions of the genomic DNA extraction kit: (1) Buffer ATL was added in a EP tube by a ratio of 1:1 to lyse tissue cells; (2) 20 μl of proteinase K was drawn and added into the EP tube; (3) 200 μl of sample was added and mixed thoroughly; (4) 200 μl of Buffer AL was added and fully mixed by vortexing shaking for 15 s; (5) incubation was made at 56° C. for 10 min; (6) centrifuge was made at 10000 rpm for 2 min to collect supernatant; (7) 200 μl of anhydrous ethanol was added and fully mixed by vortexing shaking for 15 s, so as to take supernatant; (8) the supernatant was transferred to an adsorption column, and centrifuged at 8000 rpm for 1 min; (9) the filtrate and the collection tube was removed, the adsorption column was placed into a new collection tube, and 500 μl of Buffer AW1 was added, and centrifuged at 8000 rpm for 1 min; (10) the filtrate and the collection tube was removed, the adsorption column was placed into a new collection tube, and 500 μl of Buffer AW2 was added, and centrifuged at 14000 rpm for 3 min; (11) the filtrate and the collection tube was removed, the adsorption column was placed into a new collection tube, and centrifuge was made at 14000 rpm for 1 min; (12) the filtrate and the collection tube was removed, the adsorption column was placed into a new collection tube, and a cover was opened for 3 minutes; (13) 30 μl of BufferAE was dropped into the adsorption column in suspension, stood at a room temperature for Imin, centrifuged at 8000 rpm for 1 min, and stored at −20° C.

### 2. PCR Amplification

### (1) Design and Synthesis of Primer

A gene sequence (NC_000919) of Nichols Houston strain TP0136 was queried from genbank (https://www.ncbi.nlm.nih.gov/genbank/). According to a primer design principle and combined with an enzyme-digestion site of a vector plasmid pNLF1-N, the primer was designed by a Primer Premier 6.0 software according to the primer design principle, and the primer was synthesized by Shanghai Sangon Bioengineering Co., Ltd.

### (2) PCR Amplification

Using DNA of the Treponema pallidum Nichols Houston strain as the template and Nichols Houston strain TP0136 as the primer, a total volume of a PCR reaction was 20 μl, and an amplification system was as follows:
1) 0.2 μl of DNA template
2) 0.4 μl of Upstream Primer (P1)
3) 0.4 μl of Downstream Primer (P2)
4) 9 μl of ddH₂O
5) 10 μl of PrimeSTAR® HS DNA Polymerase

A total volume of the amplification system is 20 μl. Above operations need to be carried out on ice, and then briefly centrifuge was made after vortexing shaking and mixing. PCR amplification parameter setting: predegeneration at 94° C. for 5 min; degeneration at 94° C. for 1 min, annealing at 60° C. for 2 min and extending at 72° C. for 1 min, with a total of 45 cycles; extending at 72° C. for 10 min; and then termination of reaction. 5 μl of amplification products was taken, and amplification results were detected by 1.0% agarose gel electrophoresis, and the remaining products were stored in a refrigerator at −20° C. for later use.

### (3) Preliminary Identification of PCR Amplification Products

5 μl PCR products were added into 1 μl of 6×loading buffer, which was then evenly mixed, subjected to the 1.0% agarose gel electrophoresis at a voltage of 100 V, and then placed in a gel imaging system for observation after electrophoresis, with the results being saved.

3. Construction of Eukaryotic Expression System pNLF1-N-TP0136

Purified and recovered PCR amplification products of Nichols Houston strain TP0136 were ligated with pNLF1-N vector after subjected to EcoR I and Xba I double digestion respectively, and the recombinant plasmid pNLF1-N-TP0136 was constructed.

(1) The pNLF1-N Plasmid was Subjected to Double Digestion with an Enzyme Digestion Reaction System of 40 μl, and the Reaction System was as Follows:
1) 12 μl of pNLF 1-N
2) 2 μl of Eco R I
3) 2 μl of Xba I
4) 4 μl of 10×Buffer
5) 20 μl of ddH₂O

A total volume of the reaction system is 40 μl. It was shaken and mixed well, then placed in a water bath to react for 2 h.

(2) pNLF1-N was Subjected to Eco R I and Xba I Double Digestion with an Enzyme Digestion Reaction System of 40 μl, and the Reaction System was as Follows:
1) 12 μl of pNLF1-N
2) 2 μl of Eco R I
3) 2 μl of Xba I
4) 4 μl of 10×Buffer
5) 20 μl of ddH₂O

A total volume of the reaction system is 40 μl. It was shaken and mixed well, then placed in a water bath to react for 2 h.

(3) PCR Recovered Products were Subjected to Double Digestion, with a Enzyme Digestion Reaction System of 40 μl, and the Reaction System was as Follows:
1) 18 μl of Nichols Houston strain TP0136 recovered products
2) 2 μl of Eco R I
3) 2 μl of Xba I
4) 4 μl of 10×Buffer
5) 14 μl of ddH₂O

A total volume of the reaction system is 40 μl. It was shaken and mixed well, then placed in a water bath to react for 2 h.

After enzyme digestion, a target fragment was recovered by a DNA purification and recovery kit, and operation steps followed instructions of the kit.

The concentration of the recovered target fragment product was detected by a spectrophotometer. With a molar ratio of a carrier to the target fragment to be ligated of 1: 6 and 15 μl of the reaction system, the reaction system was as follows:
1) 5.5 μl of pNLF1-N subjected to enzyme digestion
2) 2 μl of target fragment subjected to enzyme digestion
3) 7.5 μl of DNA Ligation solution

A total volume of the reaction system was 15 μl, which was shaken and mixed evenly and ligated at 16° C. for 12 hours.

### 4. Transformation of Ligation Products

(1) 100 μl of competent cells of E. coli DH5α was taken from a refrigerator at −80° C., which was then placed in ice;
(2) the water bath was preheated to 42° C.;
(3) 5 μl of the ligation products were added to the competent cells of E. coli DH5α, which is then gently mixed evenly, and incubated on ice for 30 min;
(4) the competent cells were subjected to heat shock at 42° C. for 90s;
(5) they were then transferred to the ice immediately for 2 min;
(6) 800 μl of non-resistant LB liquid culture medium preheated at 37° C. was added;
(7) it was resuscitated in a shaker at 37° C. and 200 rpm for 1 hour;
(8) in an ultra-clean workbench, 100 μl of the culture medium was smeared evenly on the LB medium containing ampicillin, which was then incubated upside down at 37° C. overnight.

### 5. Screening and Identification of Recombinant Bacteria

(1) 10 single colonies were picked and inoculated in 500 μl of LB liquid medium containing AMP, and cultured for 5 hours in a shaker at 37° C. and 150 rpm;
(2) PCR identification of recombinant bacteria: a transformant was verified by PCR amplification, with an amplification system as follows:
1) 1 μl of bacterial liquid
2) 0.2 μl of Upstream primer (P1)
3) 0.2 μl of Downstream primer (P2)
4) 5 μl of Taq DNA Polymerase
5) 3.6 μl of ddH₂O

A total volume of the system was 10 μl.

PCR amplification parameter setting: predegeneration at 94° C. for 5 min; degeneration at 94° C. for 1 min, annealing at 60° C. for 2 min and extending at 72° C. for 1 min, with a total of 45 cycles; extending at 72° C. for 10 min; and then termination of reaction. 5 μl of amplification products was taken, and amplification results were detected by 1.0% agarose gel electrophoresis. 5 μl PCR products were added into 1 μl of 6×loading buffer, which was then evenly mixed, subjected to the 1.0% agarose gel electrophoresis at a voltage of 100 V. 5 μl of correctly transformed bacterial liquid was inoculated into the LB liquid medium containing AMP, and cultured at 37° C. and 200 rpm overnight.
(3) Extraction of recombinant plasmid: operations were made according to instructions of the plasmid extraction kit: 1) 5 ml of bacterial liquid was taken and centrifuged at 12000 rpm for 1 min, and supernatant then was removed; 2) 150 μl of a P1 solution was added into a EP tube filled with cell precipitation, and shaken and mixed evenly; 3) 150 μl of a P2 solution is added into the EP tube, which was then gently turned over to lyse the cell; 4) 300 μl of a P5 solution was added into the EP tube, which was then immediately turned gently to make it fully mixed, centrifuged at 12000 rpm for 2 min; 5) supernatant was collected and added into an adsorption column CP3 and centrifuged at 12000 rpm for 30 s, and a waste liquid in a collection tube was removed; 6) 300 μl of a rinsing liquid PW was added into the adsorption column CP3 and centrifuged at 12000 rpm for 30 s, and the waste liquid in the collection tube was removed, and then this step was repeated; 7) the adsorption column CP3 was placed into the collection tube and centrifuged at 12000 rpm for 2 min; 8) the adsorption column CP3 was opened and placed at a room temperature for 5 min to dry residual rinsing liquid in the adsorption column; 9) the adsorption column CP3 was placed in a new centrifuge tube, and 50 μl of an elution buffer EB was dropped into a middle of a adsorption membrane in suspension, which was then placed at the room temperature for 5 min and centrifuged at 12000 rpm for 2 min, and a solution obtained after centrifugation is added into the adsorption column again, which is centrifuged again to increase recovery efficiency.
(4) The recombinant plasmid was identified by Eco R I and Xba I double digestion.

### 6. Sequencing

Base sequences of the recombinant plasmid pNLF1-N-TP0136 was detected by Shanghai Sangon Biotechnology Co., Ltd. Sequencing results were aligned to published gene sequences with BLAST (https://blast.nebi.nlm.nih.gov/Blast.cgi).

### 7. Cell Transfection (Liposome-Mediated Method)

(1) He La cells were inoculated one day before transfection, and transfection was performed when cell fusion degree reached 80%;
(2) 50 μl of a solution A contains 4 μl of Lipofectamine 3000 and 46 μl Opti-MeM, and 50 μl of a solution B contained 3 μl of P3000 and recombinant plasmid and Opti-MeM;
(3) the solution A and solution B were mixed, and stood for 5 min;
(4) the mixed A and B solution and 900 μl of Opti-MeM were added per well, which is then mixed evenly and cultivated at 37° C. for 48 h.

### 8. Preparation of Cell Lysate:

(1) the culture solution was drawn from a cell well to be tested, which was then rinsed carefully with PBS for 2 times and digested with Trypsin at 37° C. for 2 min, and digestion was terminated at 1 ml of culture solution;
(2) cells were drawn into a centrifuge tube and centrifuged at 1500 rpm for 5 min, and then supernatant was removed;
(3) 500 μl of PBS was added for rinsing, which was centrifuged at 12000 rpm for Imin, and supernatant was removed;
(4) 150 μl/well of lysis solution was added, stood for 30 min, centrifuged at 12000 rpm for 4 min, and the supernatant was taken. Cell lysate was stored at −80° C.

### 9. Coated Plate:

(1) 50 μl of Protein G (5 μg/ml) was added to each well of a 96-well whiteboard, which was coated at 4° C. overnight. The whiteboard was washed with PBST once.
(2) Sealing: 300 μl of sealing liquid (5% skim milk) was added to each coated well, and incubated at 37° C. for 1 h.

### 10. Detection Operation Flow of the Kit is as Follows:

(1) sample addition: 50 μl of cerebrospinal fluid of neurosyphilis patients was added into each well, and each sample was repeated for 3 wells, and positive control, negative control and blank control are set at the same time;
(2) incubation: the incubation was made at 37° C. for 1 h;
(3) board washing: the board was washed with washing liquid for 5 times;
(4) addition of pyrolysis liquid: 50 μl of Nichols Houston strain TP0136 lysate diluted with a sample diluent at a ratio of 1:100 was added to each well;
(5) incubation: the incubation was made at 37° C. for 30 min;
(6) board washing: the board was washed with washing liquid for 5 times;
(7) color development: with shielding light, 50 μl of furizine luciferase substrate was added into each well;
(8) Interpretation of results: sample fluorescence values (light units, LU) are read using a Gaomax fluorescence photometer.

### 11. Evaluation of Diagnostic Efficiency of the Kit of the Present Disclosure

According to clinical symptoms and signs of patients and results of serum, spine and brain examination, 200 syphilis patients were divided into a neurosyphilis group, a suspected neurosyphilis group and a non-neurosyphilis group, in which 46 cases were into the neurosyphilis group, 23 cases were into the suspected neurosyphilis group and 131 cases were into the non-neurosyphilis group, and cerebrospinal fluid from the three groups of patients was tested by the kit of the disclosure. Diagnostic criteria for the neurosyphilis patients are syphilis patients with positive results in VDRL tests in cerebrospinal fluid at any stage. Diagnostic criteria for the suspected neurosyphilis patients are syphilis patients at any stage with negative results in VDRL tests and satisfying following two items: (1) FTA-ABS for cerebrospinal fluid is positive and (2) a CSF WBC count for the cerebrospinal fluid is increased (>5/μl) or protein concentration for the cerebrospinal fluid is increased (>45 mg/dl) or there are neurological symptoms consistent with neurosyphilis with a case of being caused by other diseases excluded. The results showed that: (1) the Nichols Houston strain TP0136 antibody level in the cerebrospinal fluid was tested by Kruskal-Wallis H test as follows: neurosyphilis > suspected neurosyphilis > non-neurosyphilis, with statistical difference among them; (2) an optimum cutoff value was determined according to a maximum Youden Index (sensitivity+specificity−1), and when a cutoff value of the Nichols Houston strain TP0136 antibody level in the cerebrospinal fluid was selected to be greater than 7, an area under the curve (AUC) was 0.898, sensitivity was 75.4%, specificity was 89.3%, a positive predictive value (PPV) was 87.3%, a negative predictive value was 78.8%, and an agreement (Agreement) is 84.5%. (3) Using a logistic regression equation and combined with patients' age, sex, presence or absence of nervous system symptoms, serum TRUST and the Nichols Houston strain TP0136 antibody level in the cerebrospinal fluid, an accuracy of neurosyphilis prediction can be further improved to 93.3%. A prediction formula is: In (P/1-P)=−16.840+0.06×age+1.009×sex+3.967% presence or absence of nervous system symptoms+0.082×serum TRUST+1.621×Nichols Houston strain TP0136 antibody level in cerebrospinal fluid. According to the prediction formula, the sensitivity is 89.2%, the specificity is 95.6%, the positive predictive value (PPV) is 92.1%, the negative predictive value (NPV) is 94.0%, and the agreement (Agreement) is 93.3%.

It is unexpectedly found in this disclosure that detecting the Nichols Houston strain TP0136 antibody level for diagnosis of neurosyphilis makes up a blank of diagnosis and detection methods of the neurosyphilis. The method has such unexpected technical effects of high sensitivity and high prediction accuracy, has characteristics of large detection throughput, simple operation and easy popularization, and has market value.

The basic principles, main features and advantages of the present disclosure are shown and described in above. It should be understood by those skilled in the industry that the above embodiments do not limit the present disclosure in any form, and all technical solutions obtained by equivalent substitution or equivalent transformation fall within the protection scope of the present disclosure.

What is claimed is:

1. A fusion protein for detecting neurosyphilis, wherein the fusion protein is for expressing a gene fragment of a Nichols Houston strain TP0136 antigen and a bioluminescence reporter gene, the gene fragment of the Nichols Houston strain TP0136 antigen is a fragment of a Gene group sequence with genbank ID of 3322413, located from Start: 156807 to Stop: 158276 and with Gene Length:1470; and the gene fragment of the Nichols Houston strain TP0136 antigen is obtained by taking DNA of Treponema pallidum Nichols Houston strain as a sequence template and taking the Nichols Houston strain TP0136 as a primer.

2. The fusion protein for detecting the neurosyphilis according to claim 1, wherein the sequence template SEQ01 is 16 to 1470 sites of the gene fragment of the Nichols Houston strain TP0136 antigen; and an upstream primer SEQ02 is 5′-ATGACGTGCGATTTCACTGG-3′, and a downstream primer SEQ03 is 5′-CTCGCGGTTCCAGGAGCACG-3′.

3. The fusion protein for detecting the neurosyphilis according to claim 1, wherein the bioluminescence reporter gene is a luciferase reporter gene.

4. The fusion protein for detecting the neurosyphilis according to claim 3, wherein the luciferase reporter gene is a dual-luciferase reporter gene.

5. The fusion protein for detecting the neurosyphilis according to claim 1, wherein the fusion protein is obtained by expression of a recombinant plasmid obtained by respective enzyme digestion and ligation of the gene fragment of the Nichols Houston strain TP0136 antigen and a pNLF1 vector plasmid of a NanoLuc luciferase gene.

6. The fusion protein for detecting the neurosyphilis according to claim 5, wherein the enzyme digestion is EcoR I and Xba I double digestion, with a ligase being a T4 ligase.

7. The fusion protein for detecting the neurosyphilis according to claim 5, wherein the expression is made by following steps:
1) transforming the recombinant plasmid into an E. coli DH5α competent cell, and then incubating, inoculating, culturing, PCR identifying and sequencing;
2) performing cell transfection on the recombinant plasmid by using a liposome-mediated method; and
3) performing cell lysis on transfected cells and then collecting supernatant which is diluted to obtain the fusion protein.

8. A fusion protein kit for detecting neurosyphilis, comprising: (1) a sample diluent; (2) a fusion protein solution consisting of NanoLuc luciferase and a Treponema pallidum Nichols Houston strain TP0136 antigen; (3) a protein A/G coated enzyme-labeled plate; (4) a washing liquid; and (5) a luciferase substrate.

9. The fusion protein kit for detecting the neurosyphilis according to claim 8, wherein the Treponema pallidum Nichols Houston strain TP0136 antigen is extracted by inoculating Treponema pallidum Nichols Houston strain into testes of 3-month-old New Zealand rabbits, and then extracting a genome of a testicular tissue sample with a DNA extraction kit.

10. The fusion protein kit for detecting the neurosyphilis according to claim 8, wherein the sample diluent is 2% skim milk.

11. The fusion protein kit for detecting the neurosyphilis according to claim 8, wherein the washing liquid is with a formula of: 8 g of NaCl, 0.2 g of KCl, 3.63 g of Na₂HPO₄·12H₂O, and 0.24 g of KH₂PO₄, with a volume being adjusted to 1 L by adding water.

12. The fusion protein kit for detecting the neurosyphilis according to claim 8, wherein the luciferase substrate is a furimazine luciferase substrate from Promega Corp.


SEQUENCE LISTING (5 sequences)

SEQ ID NO: 1 - DNA, 1470 bp, Treponema pallidum
  source 1..1470: mol_type = genomic DNA
atggatacgcagtatatgaggcgccgggtgtgcacggtggtgcgcgcggtggtgtgtctactcagcacgagtttgctgaccacgtgcgatttcactggcatctttgcggcaattcagtcggaagtgcccattaaaacgccgtccatcccgggggcgatttatggcctggtcaaggccgggagcaagctctacgccaccaacggccggctttgggaaaaggagctgaacggcactgggtcgtggcagaaagtgtcttcctcgtccgttcccactgactcggataaaaaggttatgagcattgccaccgacgggaacacgttcgtcctcgcctgcgtgcctggcacgggcgtttacaaacactgcgtaaatggcgcgggcagctcaagcaccggcacaacggcaagcccctcgactgaaacctgctcgcagcatgcgacgctcgtggggggaacgtccaagcccttctggctcgtgccgggaggcacggggaataatgggaactgcggttgcgggggaggggggggtggctcctcctcgagtagcagctcgtgcattcacatctggctcgtgccgggaggcacggggaataatgggaactgcggttgcgggggaggggggggtggctcctcctcgagtagcagctcgtgcattcacattaaggtagaaaacacggacgaacagtttctcgatatgggtgaggggtacgtggtgaccaccaagcacctctacaccaaaaacggctcgtccagcgcgggaccggcgcagtgtcccggtggcggtggcggcggaggcagcagcgggggtgggggttcctcggagtacaccaaagcttcctgttccttttccacgcccattctggcaagcgtcagcgacgggtgctatcactacattctcaccaaagaaaaagtgtactgcagaaagcaggacaccgcttcctccgctgcgtcgtcaccagcccagtgtccctcttccccttcttcttcttcctcctcctcgacgaatgcgggatgcgaggtggcgcacggggtggacgacccgctgtgtcttgcgatttttaaacacaacggctgcgaatacttgctcatcggcggcagtcggggctacggggaaataaagctggaagcgaactccagcggtacgaacggcacctgcatgcgattgaaagagagcaatgtgcacaagagtccgggccagtggggcgagtcgagccccacgcccaaagcgagcgccgagcagtatcggggcacggtcggtcggtttgccgtgcagaaaatctacgtagttgaaaaaaatggcggtgggaacggtgtcgccgcgggtggggcgggctgtcctgcaaacgccagcagttccagcggagggaccagcagcacgcagcgtccagacctctacgccgcagtgggggagtcgagcgacacctacacggggctgtggaagtttgacaccaccacgtgctcctggaaccgcgag

SEQ ID NO: 2 - DNA, 20 bp, synthetic construct
  source 1..20: mol_type = other DNA
atgacgtgcgatttcactgg

SEQ ID NO: 3 - DNA, 20 bp, synthetic construct
  source 1..20: mol_type = other DNA
ctcgcggttccaggagcacg

SEQ ID NO: 4 - DNA, 20 bp, synthetic construct
  source 1..20: mol_type = other DNA
atgacgtgcgatttcactgg

SEQ ID NO: 5 - DNA, 20 bp, synthetic construct
  source 1..20: mol_type = other DNA
actacgtagattttctgcac